(19) United States
(12) Patent Application Publication
(10) Pub. No.: US 2017/0225171 A1
(43) Pub. Date: Aug. 10, 2017

(54) PROCESS & APPARATUS FOR REACTIONS

(71) Applicant: BG RESEARCH LTD, Kimbolton, Cambs (GB)

(72) Inventors: Nelson Nazareth, Upper Dean (GB); David Edge, Warlingham (GB); Adam Tyler, Burton Latimer (GB)

(21) Appl. No.: 15/330,039

(22) PCT Filed: Jan. 28, 2015
(86) PCT No.: PCT/GB2015/000030

§ 371 Date: Jul. 29, 2016

(30) Foreign Application Priority Data
Jan. 29, 2014 (GB) 1401584.6

(51) Int. Cl. B01L 9/00; G01N 21/01; C12Q 1/68; G01N 21/64; B01L 3/00; B01L 7/00

(52) U.S. Cl. CPC B01L 9/523; B01L 3/50851; B01L 7/52; C12Q 1/686; G01N 21/6428; G01N 21/6452; G01N 21/6456; G01N 21/01; B01L 2200/025; B01L 2200/028; B01L 2300/0654; B01L 2300/0672; B01L 2300/0829; B01L 2300/1822; B01L 2300/1827; B01L 2300/185; B01L 2400/0421; G01N 2021/6439

(57) ABSTRACT

A heat removal module slice constructed to service a row of reaction vessels, the slice being in the form of a block of thermally conductive material having a row of reaction stations at an edge thereof, at one end thereof a liquid entry manifold and at the other end thereof a liquid exhaust manifold; and a heat exchanger liquid channel adjacent the reaction stations and extending between the two manifolds. The slice is constructed to form, with a plurality of similar slices, a heat reduction module for incorporation in a reaction, typically a PCR reaction, apparatus and process.

### FIELD OF THE INVENTION

The present invention relates to biological, chemical and biochemical reactions, particularly those carried out at the nanolitre to microlitre level, and may even include those carried out at the picolitre level. It includes those involving thermal cycling such as polymerase chain reactions (PCR) as well as isothermal reactions.

It is further particularly concerned with apparatus in which a large number of reduced volume reactions are carried out simultaneously, with a plurality of reaction vessels being received in a reaction apparatus at one time. At the microlitre level, for example the reaction vessels may be in the form of a tray, known as a microtitre plate, comprising an array of vessels. In one standard microtitre plate, 96 vessels are set out in one array comprising 12×8 rows. Other plates are then normally constructed on a 96×n basis, where n is an integer.

### BACKGROUND TO THE INVENTION

Particularly in the field of PCR, where it can be valuable to effect a complete reaction in the minimum possible time, the rates at which heat can be both transferred into and out of a sample are important. This implies not only consideration of the heat transfer media and optimum base temperatures but also the proximity of the heating and cooling media to the sample. In the context of a 96 n microtitre array where it is also particularly desirable to have individual control of the reaction in each vessel, if, as may be preferred, the cooling is by means of a single block operating at a base temperature then it is vital to ensure that the same base temperature is consistently available to each vessel.

One such single block is a heat removal module (HRM) as described in PCT Patent Application PCT/GB07/003564. The module is a single block having a labyrinthine channel formed therein wherethrough coolant can flow. The module is formed to receive microtitre reaction vessels. However whilst in the system described in that Patent Application the cooling facility is fairly efficient the heating facility is, on the other hand, less so.

PCT Patent Application WO2012063011 describes a reaction vessel receiving station having a reaction vessel receiving portion; a heater portion and a cooling portion, the latter being arranged to anchor the station in a heat removal module. The heater portion, comprising a wire wrapped around the vessel receiving portion is particularly efficient.

The present invention provides a heat removal system which meets the requirements for consistent cooling from each reaction vessel.

### SUMMARY OF THE INVENTION

According to the present invention there is provided a heat removal module slice constructed to service a row of reaction vessels, the slice being in the form of a block of thermally conductive material having a row of reaction stations at an edge thereof, at one end thereof a liquid entry manifold and at the other end thereof a liquid exhaust manifold; and a heat exchanger liquid channel adjacent the reaction stations and extending between the two manifolds.

The reaction vessel receiving stations preferably define recesses into which reaction vessel holders can be mounted, preferably as an interference fit.

According to a feature of the invention, with the manifolds extending from one face of the slice to the other, a slice may be constructed for assembly face to face into an array of similar such slices, so that the manifolds of each form continuous manifold entry and exit tubes, and each slice may incorporate locating and attachment means whereby slices may be correctly located and attached one to another.

Important advantages of forming a heat removal module by the assembly of a plurality of slices as defined are ease of manufacture, obtaining efficient and consistent cooling to each reaction station, and relatively inexpensive removal and replacement of a component, e.g. a slice in the event of failure of a reaction vessel receiving member. In a 12×8 well array system it is preferred that the slice is constructed to service a row of eight stations.

Bearing in mind that the area above a heat reduction module can be quite congested, another advantage associated with the facility of forming a heat removal module from slices is that a slice can be manufactured to incorporate grooves for electrical conduits for attachment to reaction vessel holders, for both powering heaters thereof and conveying sensor, such as temperature sensor, signals therefrom. These conduits can be formed on printed circuit boards (PCBs), indeed PCBs constructed to fit, ideally to click, in the grooves. This can also facilitate manufacture of a heat reduction module because with reaction vessel holders mounted in the stations, each incorporating a heater and a temperature sensor, and a dedicated PCB in place, the connection of the heater and the sensor to the conduits can be relatively easy. Typically the conduits terminate in fine tubes into which the sensor and heater leads can be fed and soldered or simply clamped (crimped) in place.

In the manufacture of a slice, having first of all cut the shape, formed the necessary holes and milled the grooves for the PCB and, with the slice held in a jig with a suitable former against the side thereof opposite the grooves, fitted the vessel holders, the PCB is then clipped in place and the vessel holder sensor and heater wires attached to the PCB conduit terminals. Then silicone can be fed around the vessel holders to insulate the vessel holder heater coil and to assist in maintaining integrity. To isolate thermally as far as possible, each station one from the other gaps, for example cuts, may be formed between each station of a slice, and the slice may be rebated with respect to an adjacent slice.

A typical standard microtitre 12×8 plate is constructed with well centres at 9.00 mm centres. The reaction vessel is a microtitre vessel formed of a carbon loaded plastics material and is 2 cm overall length. It comprises, in descending order, a cap receiving rim, a filler portion and a reaction chamber with a base thereto. The filler portion has a maximum outer diameter of 7 mm and a depth of 5 mm. The reaction chamber tapers down from 3 mm to 2.5 mm, the whole having a wall thickness of 0.8 mm. Accordingly the reaction vessel is of substantially capillary dimensions.

Thus a HRM slice may be 9.00 mm thick. To incorporate 14.00 mm manifolds and their associated connectors to (preferable flexible) coolant pipes, a slice may be 11-12 cm long and 4-5 cm deep. The heat exchanger liquid channel may have a bore of about 3-4 mm. Typically a slice is formed from relatively pure aluminium. Such aluminium is readily machinable and has a high enough thermal conductivity whilst being adequately resistant to mechanical deformation compared for example to copper and plastics material and cheaper than say stainless steel. Aluminium is also easily protectable by anodisation and adequately resistant to oxidization.

It will be appreciated then that a standard HRM module will comprise twelve HRM slices plus end clamping members incorporating the coolant pipe connectors.

Such a HRM is typically mounted in a reaction apparatus where it may be movable between loading and operating stations. The loading station may project from the apparatus where the module can receive a microtitre plate loaded with ninety six reaction wells charged with reaction components. A motor then retracts the module and lifts it to an operation station where mechanical pressure causes contact to be maintained between each well and its vessel holder while the desired reaction takes place. The apparatus may incorporate sensing means for indicating that the desired contact pressure has been achieved and maintained. The reaction apparatus will normally also have a facility, typically an optical facility, arranged for monitoring the outcome of the reaction.

During a reaction electrical supply via the conduits may be arranged to heat the wells according to a predetermined program, while other of the conduits convey signals relating to the temperature in the wells.

The heating cycle may be arranged to take place against a coolant environment in the HRM 50 which is preferably fixed somewhat above room temperature, for example between 30 and 45° C. Having a higher HRM temperature allows higher heating rates to be achieved—to the typical maximum of 96° C. Conversely, the lower the HRM temperature the faster the cooling rate will be. A desirable mean is 40° C. which is usually above room temperature and is a mid-point for heating and cooling efficiency.

This apparatus is particularly suited to the individual control of the reaction cycle in each well.

### BRIEF DESCRIPTION OF THE DRAWINGS

Embodiments of the invention will now be described by way of example with reference to the accompanying drawings, of which:

FIG. 1 is an isometric view of a heat reduction module slice;

FIG. 2 is an isometric view of a slice with a fitted PCB;

FIG. 3 is an isometric view of a slice with fitted PCB and reaction vessel holders;

FIG. 4 is a face view of a slice fitted with a PCB and showing the location and structure of a reaction vessel holder;

FIG. 5 is a plan view of an assembled HRM;

FIG. 6 is a schematic view of a reaction apparatus; and

FIGS. 7 and 8 are isometric views of an alternative slice.

### DESCRIPTION OF EMBODIMENTS OF THE INVENTION

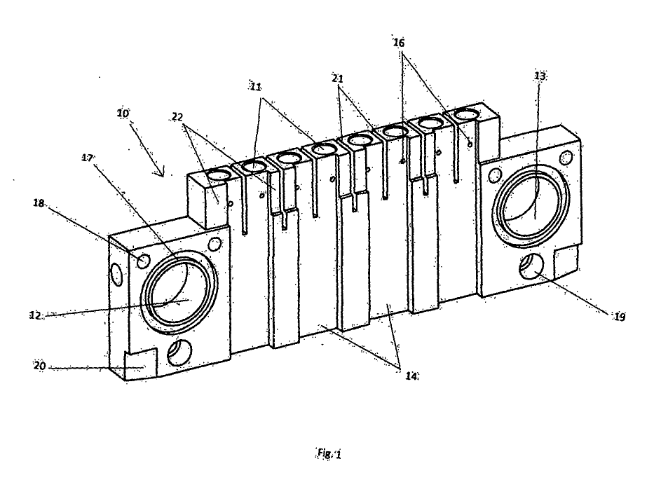

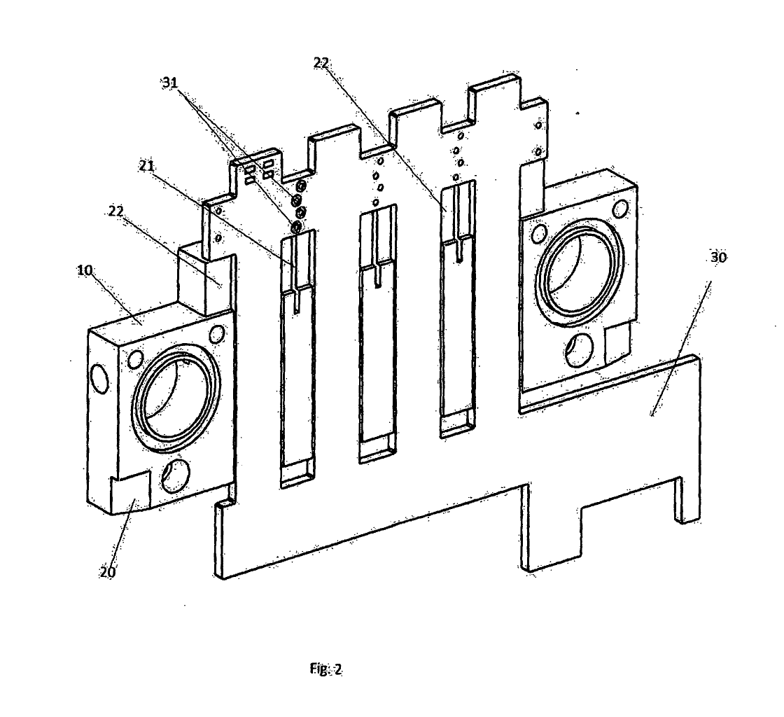

Shown in FIGS. 1 to 5 is a heat removal module slice 10. Formed of aluminium it has a plurality of reaction stations 11 at a top edge, coolant liquid entry 12 and exit 13 manifold bores therethrough at each end, and a series of grooves 14 extending along one face from the top to the bottom edge thereof. A heat exchanger liquid channel 15 extends between the manifold bores adjacent the reaction stations 11.

The reaction stations 11 are circular hollows sized for the bases of reaction vessel holders 40 to be an interference fit therein. A small hole 16 leads from the base of each station 11 to the groove 14 and acts in use to permit the escape of gases (air) from the stations 11 when the vessel holders are driven in.

Around each manifold on one face of the slice are grooves 17 for an O-ring seal and further out are slide attachment holes 18 of which one has a locating bush 19.

At each bottom corner on one face is a separation rebate 20 arranged to assist in separating the slices when required. Between each station 11 there is a cut 21 arranged to maximise thermal isolation between each station 11. Rebates 22 on one side of each slice 10 are formed for a like purpose.

A printed circuit board (PCB) 30 is manufactured to clip into the grooves 14 and project above and below the slice 10. The PCB 30 carries heater and sensor electrical conduits which terminate in connectors 31 at the top and 32 at the bottom thereof. The breadth of the PCB 30 is the depth of the grooves 14.

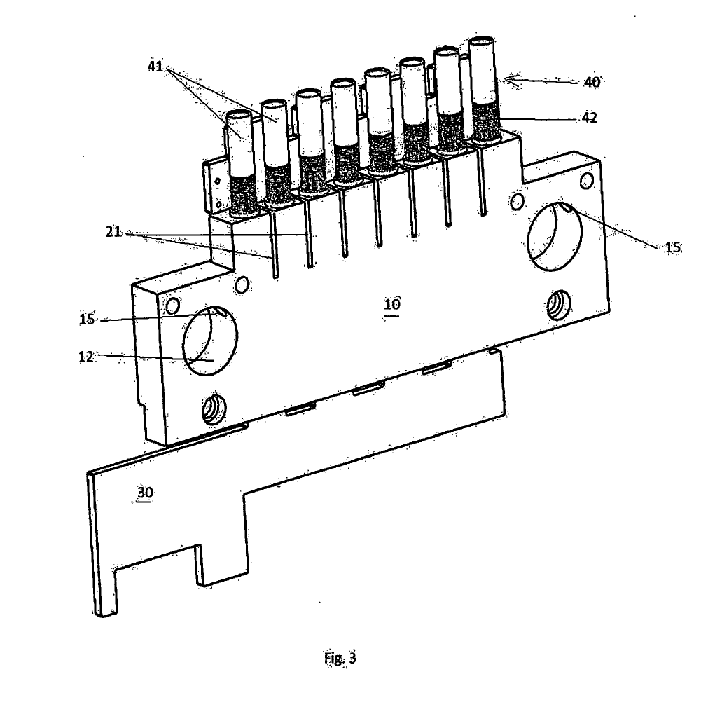

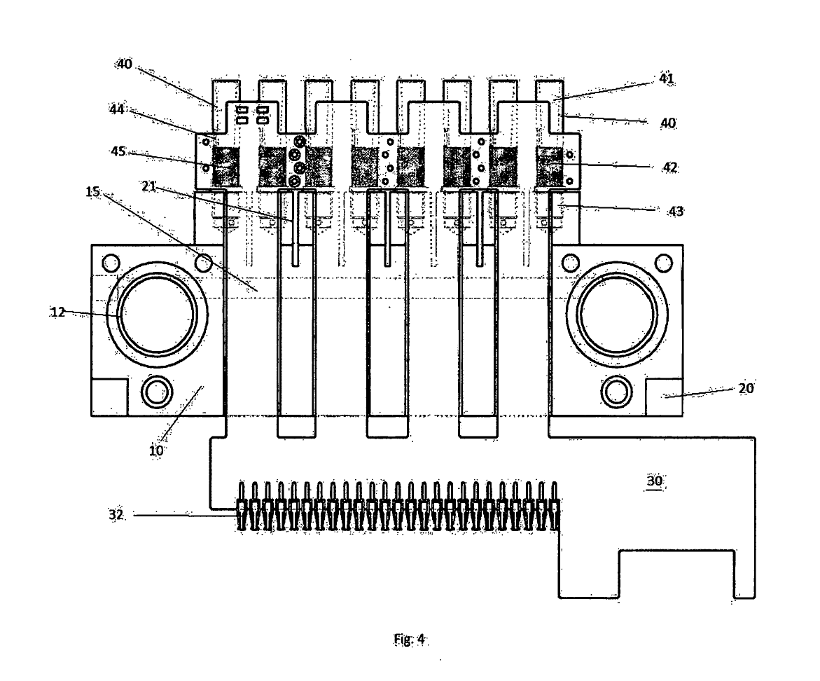

As shown particularly in FIGS. 3 and 4, a reaction vessel holder 40 fits into each of the reaction stations 11. The reaction vessel holder 40 comprises a reaction vessel receiving portion 41; a heater portion 42 and a cooling portion 43, the latter being arranged to anchor the station in a heat removal module. Formed also dowel-like of aluminium the holder 40 is sized and shaped to be driven into the reaction station 11. The vessel receiving portion 41 is shaped to receive snugly a microtitre reaction vessel (not shown) and in the wall thereof is located a temperature sensor 44. The heater portion 42 has a helical groove therearound into which is laid a heater coil 45.

In the manufacture of a slice, having first of all cut the shape, formed the necessary holes and milled the grooves for the PCB and, with the slice held in a jig with a suitable former against the side thereof opposite the grooves, fitted the vessel holders, the PCB is then clipped in place and the vessel holder sensor and heater wires attached to the PCB conduit terminals.

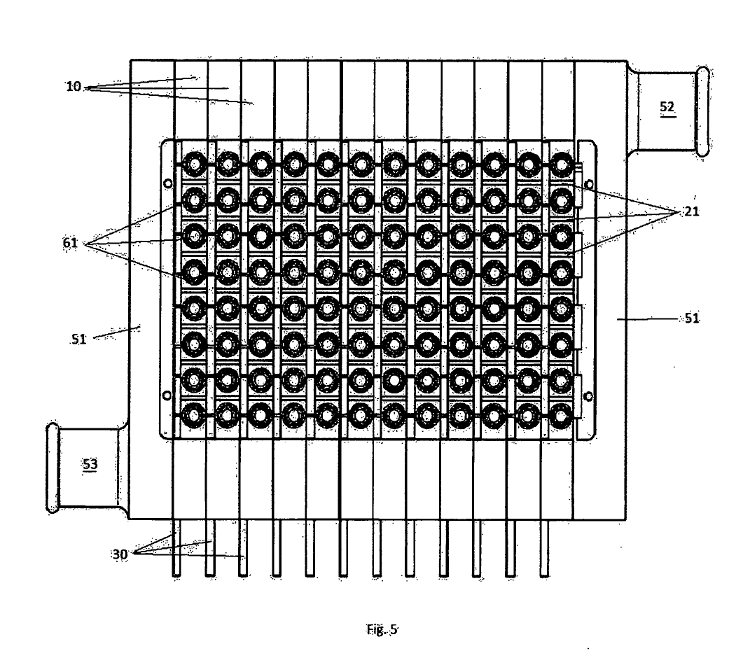

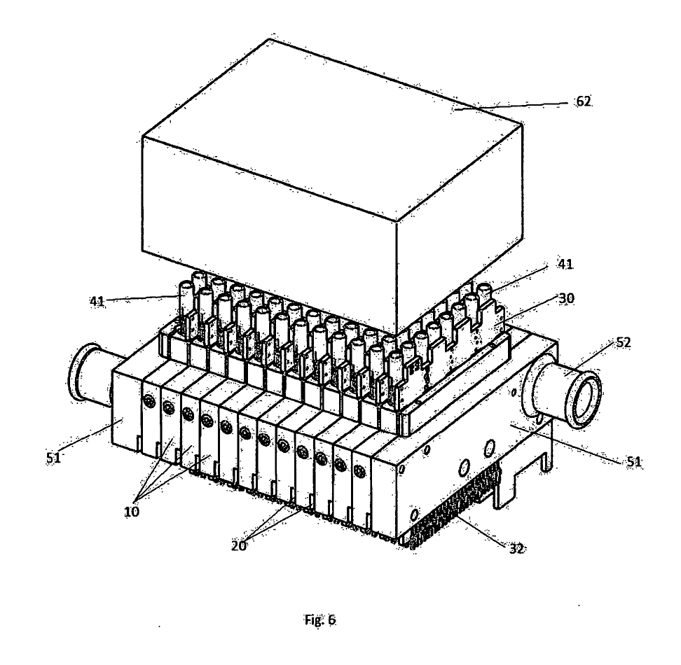

To form a heat removal module 50 for a typical 96 (12×8) well tray twelve HRM slices 10 are mounted together as shown in FIGS. 5 and 6, clamped by and between connector plates 51 having coolant liquid inlet and outlet necks 52, 53. The module 50 is incorporated in a reaction apparatus (not shown) on a motorised conveyor by which the module can be moved between a loading position, where it projects from the apparatus and an operational position within the apparatus where a reaction can take place. Flexible tubing (not shown) connects the necks 52, 53 with a heat sink coolant reservoir (not shown) via a pump (not shown).

FIG. 6 shows the assembly of a module 50 with a 96 well microtitre tray or plate 60 carrying reaction wells 61. The reaction vessel 61 is a microtitre vessel formed of a carbon loaded plastics material and is 2 cm in overall length. It comprises, in descending order, a cap receiving rim, a filler portion and a reaction chamber with a base thereto. The filler portion has a maximum outer diameter of 7 mm and a depth of 5 mm. The reaction chamber tapers down from 3 mm to 2.5 mm in diameter, the whole having a wall thickness of 0.8 mm. Accordingly the reaction vessel is of substantially capillary dimensions.

The tray 60 is adapted to be fitted onto the array of holders and the reaction apparatus is arranged evenly to press the wells into the holders. The reaction apparatus has an optical box 62 incorporating an optical facility arranged to monitor the progress of reactions in the wells 61. The optical box also functions to maintain the pressure of the wells 61 in the holders 40. The apparatus incorporates sensors (not shown) to indicate the achievement and maintenance of said even pressure.

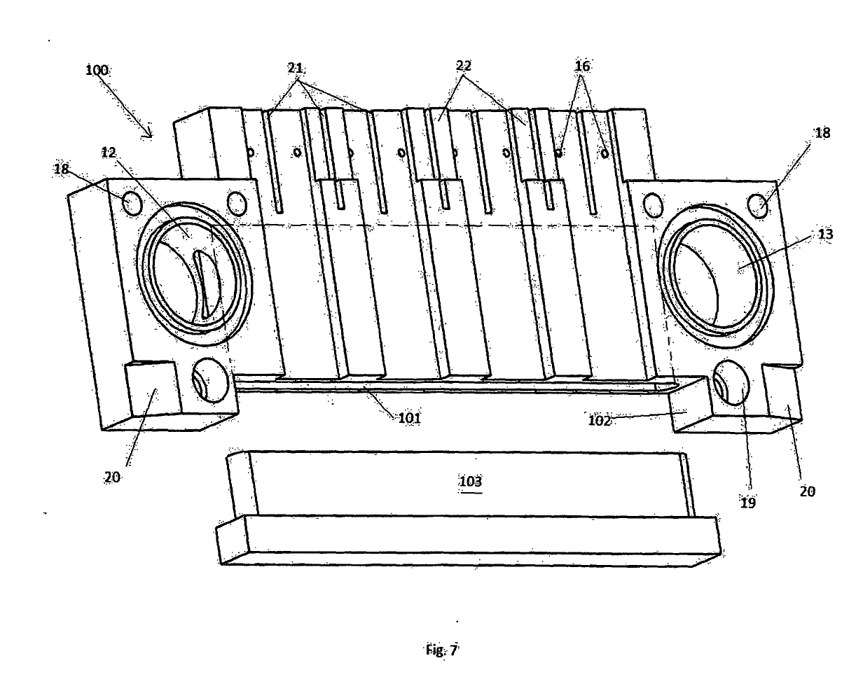

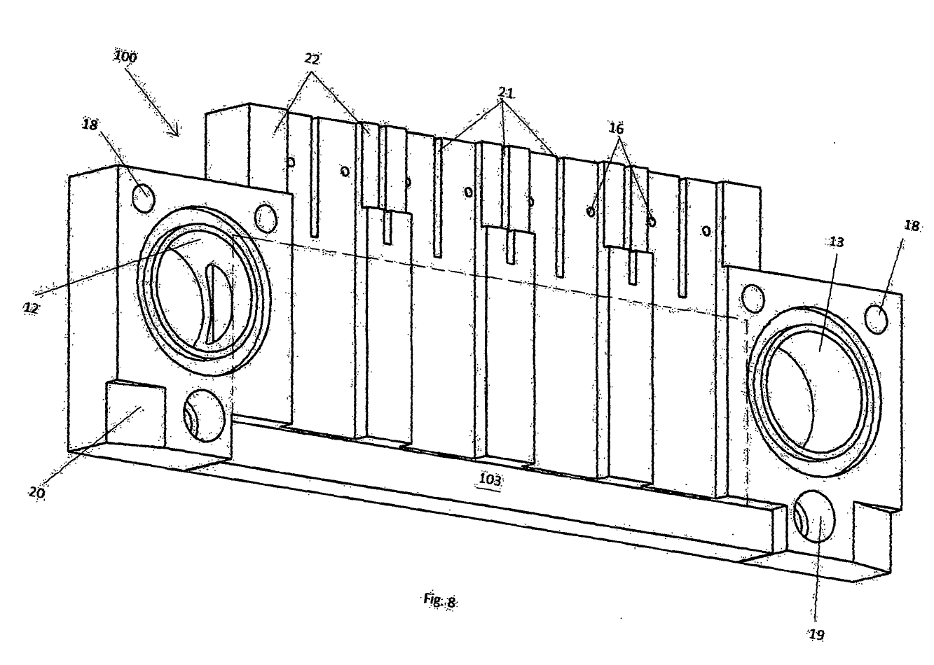

In the alternative slice 100 illustrated in FIGS. 7 and 8, like reference numbers refer to like components. The slice 100 differs from slice 10 in being formed with a rectangular hollow 101 extending from a rebated base 102 to just below the base of the stations 11 and from the entry duct 12 to the exit duct 13. A stopper 103 fitting into the rebated base 102 serves to seal the hollow 101. The hollow 101 is thus arranged to convey coolant between the entry duct 12 and the exit duct 13. The hollow 101 thus replaces the duct 15 in the slice 10 and provides for an improved coolant flow and effectiveness.

During a reaction electrical supply via the conduits is arranged to heat the wells 61 according to a predetermined program, while other of the conduits convey signals relating to the temperature in the wells. This program is predetermined for each well, as the apparatus is particularly suited for performing totally independent reactions in each well 61. Thus, where the reactions comprises a heating-cooling cycle, as is the case for example in PCR, one well 61 may be in a heating phase and another in a cooling phase, one at rest and another complete.

The heating cycle is arranged to take place against a coolant environment in the HRM 50 which is fixed at 40° C. which is usually above room temperature and is a mid-point for heating and cooling efficiency.

1. A heat removal module slice constructed to service a row of reaction vessels, comprising:
the slice being in the form of a block of thermally conductive material;
the block formed with a row of reaction vessel receiving stations along an edge thereof;
a liquid entry manifold formed at one end of the block;
a liquid exhaust manifold; formed at another end of the block spaced and opposite from the one end; and
a heat exchanger liquid channel adjacent the receiving stations and extending between, and in communication with, the entry and exit manifolds.

2. A slice as claimed in claim 1 and wherein the reaction-vessel receiving stations define recesses into which reaction vessel holders can be mounted.

3. A slice as claimed in claim 2 and wherein the recesses are arranged to receive reaction vessel holders as an interference fit.

4. A slice as claimed in claim 1 and wherein, with the entry and exit manifolds extending through from one face of the slice to the other, a slice is constructed for assembly face to face into an array of similar such slices, so that the manifolds of each form continuous entry and exit manifolds, and each slice incorporates locating and attachment means whereby slices may be correctly located and attached one to another.

5. A slice as claimed in claim 1 and constructed to service a row of eight stations in a 12×8 well array.

6. A slice as claimed in claim 1 and incorporating at least one groove for electrical conduits for attachment to reaction vessel holders, for both powering heaters thereof and conveying sensor, such as temperature sensor, signals therefrom.

7. A slice as claimed in claim 6 and having an associated printed circuit board (PCB) carrying electrical conduits and constructed to fit in the at least one groove.

8. A slice as claimed in claim 7 and wherein the conduits terminate in fine tubes into which the sensor and heater leads can be fed and soldered or simply clamped in place.

9. A slice as claimed in claim 1 and having vessel holders fitted therein.

10. A slice as claimed in claim 1 and having eight vessel holders fitted therein.

11. A slice as claimed in claim 9 and having a silicone casing around the vessel holders.

12. A HRM slice as claimed in claim 1 and which is 9.00 mm thick.

13. A slice as claimed in claim 1 and wherein the manifolds have a 14.00 mm diameter bore.

14. A slice as claimed in claim 1 and which is 11-12 cm long and 4-5 cm deep.

15. A slice as claimed in claim 1 and wherein the heat-exchanger liquid channel has a bore of about 3-4 mm diameter.

16. A slice as claimed in claim 1 and formed from pure aluminium.

17-29. (canceled)

30. A heat removal module slice constructed to service a row of reaction vessels, the slice being in the form of a block of thermally conductive material having a row of reaction-vessel receiving stations at an edge thereof, the vessel receiving stations defining recesses into which reaction vessel holders can be mounted as an interference fit; at one end thereof a liquid entry manifold and at the other end thereof a liquid exhaust manifold, the manifolds extending from one face of the slice to the other, the slice being constructed for assembly face to face into an array of similar such slices, so that the manifolds of each form a continuous entry manifold and a continuous exit manifold, a heat-exchanger liquid channel adjacent the reaction stations and extending between the entry and exit manifolds of each slice; at least one groove for electrical conduits for attachment to reaction vessel holders, for both powering heaters thereof and conveying sensor, such as temperature sensor, signals therefrom and each slice incorporating locating and attachment means whereby slices may be correctly located and attached one to another.

31. A slice as claimed in claim 30 and constructed to service a row of eight stations in a 12×8 well array.

32. A module comprising a plurality of slices, each slice being as claimed in claim 1 and end clamping members incorporating coolant pipe connectors.

33. A module as claimed in claim 32 and comprising twelve slices.

34. A reaction apparatus incorporating a module as claimed in claim 32.

35. A reaction apparatus as claimed in claim 34 and wherein the module is arranged to be movable between loading and operating stations.

36. A reaction apparatus as claimed in claim 34 arranged to receive a microtitre plate loaded with reaction vessels.

37. A reaction apparatus as claimed in claim 34 and having means to apply mechanical pressure to maintain contact between each vessel and its vessel holder while a desired reaction takes place.

38. A reaction apparatus as claimed in claim 34 and having a motor arranged to retract the module and lift it to an operation station.

39. A reaction apparatus as claimed in claim 34 and having a facility arranged for monitoring the outcome of the reaction.

40. A reaction apparatus as claimed in claim 39 and wherein the monitoring facility is optical.

41. A reaction apparatus incorporating a module as claimed in claim 32, further arranged to be movable between loading and operating stations, constructed to receive a microtitre plate loaded with reaction vessels, having a motor arranged to retract the module and lift it to an operation station, and having an optical facility arranged for monitoring the reaction.

42. A reaction apparatus as claimed in claim 41 and constructed to receive a microtitre plate loaded with reaction vessels in a 12×8 array.

43. A biological, chemical or biochemical process employing apparatus as claimed in claim 34.
